(19) United States
(12) Patent Application Publication
(10) Pub. No.: US 2026/0265685 A1
(43) Pub. Date: Sep. 10, 2026

(54) METHODS AND COMPOSITIONS FOR ISOLATING RHABDOVIRUS-FREE SF9 CELLS

(71) Applicant: Expression Systems, LLC, Davis, CA (US)

(72) Inventors: Scott Walker, Davis, CA (US); Brooks Hayes, Davis, CA (US); Lauren Hill, Davis, CA (US); Thera Mulvania, Davis, CA (US)

(73) Assignee: Expression Systems, LLC, Davis, CA (US)

(21) Appl. No.: 19/162,883

(22) PCT Filed: Mar. 15, 2024
(86) PCT No.: PCT/US2024/020112

§ 371 Date: Sep. 5, 2025

Related U.S. Application Data
(60) Provisional application No. 63/452,824, filed on Mar. 17, 2023.
(60) Provisional application No. 63/465,813, filed on May 11, 2023.

(51) Int. Cl. C12N 5/07; C12Q 1/6888; C12Q 1/70

(52) U.S. Cl. CPC C12N 5/0601; C12Q 1/6888; C12Q 1/701; C12Q 2600/158

(57) ABSTRACT

The present disclosure relates to new Spodoptera frugiperda cell lines free of Sf9 rhabdovirus, methods for making, and methods for using the same. In one aspect, the methods comprise single cell isolation to obtain clonal cell lines and confirm the absence of Sf9 rhabdovirus. In one aspect, the disclosure provides novel cell lines and kits comprising the same. In one aspect, the disclosure provides methods for using the cell lines to produce biological products.

### CROSS-REFERENCE TO RELATED APPLICATIONS

This application claims the benefit of and priority to U.S. Provisional Patent Application No. 63/452,824, filed Mar. 17, 2023, and U.S. Provisional Patent Application No. 63/465,813, filed May 11, 2023, the entire contents of which are incorporated herein by reference.

### TECHNICAL FIELD

The present disclosure relates to new Spodoptera frugiperda cell lines free of Sf9 rhabdovirus, methods and compositions for making and using the same. In one aspect, the methods comprise single cell isolations to obtain clonal cell lines and confirmation of the absence of Sf9 rhabdovirus. In one aspect, the disclosure provides novel cell lines and kits comprising the same. In one aspect, the disclosure provides methods for using the cell lines to produce biological products.

### BACKGROUND

Insect cells capable of supporting baculovirus replication are widely used in the pharmaceutical industry for the production of biological products. Spodoptera frugiperda-derived Sf9 and Sf21 cells have become mainstream for this purpose over the last several decades. The discovery that the widely used Sf9 cell line ATCC CRL-1711 is infected with a novel rhabdovirus (“Sf9 rhabdovirus”) created a substantial impediment for the production of pharmaceuticals.

It has further been reported that the ATCC CRL-1711 line is a heterogeneous population of cells comprising rhabdovirus-positive and rhabdovirus-negative cells. The line is not homogenous, which is preferred for the production of biological products.

It has further been reported that Sf9 cells tend to tetraploidy when the cells are under stress, such as when maintained under sub-optimal conditions. Tetraploid Sf9 cells perform less well than their diploid counterparts, and are disfavored for the production of biological products.

Accordingly, there is a need in the industry for homogenous, diploid Sf9 rhabdovirus-free Sf9 cell lines, and methods for making and using the same.

### SUMMARY

In one aspect, the present disclosure provides a Spodoptera frugiperda cell line deposited as accession number PTA-127562, PTA-127563, PTA-127565, PTA-127566, PTA-127567, or PTA-127568.

In some embodiments, the cell line is suspended, stored, or maintained in a preservative, cryoprotectant, solvent, buffer, media, antimicrobial agent, antibacterial agent, antifungal agent, or other agent. In some embodiments, the cell line is free of Sf9 rhabdovirus. In some embodiments, the cell line is free of nucleic acid sequences having at least 70%, 75%, 80%, 85%, 90%, 95%, 98%, or 99% homology to the sequence set forth in NCBI Accession No. KF947078.

In some embodiments, the cell line is lyophilized. In some embodiments, cells of the cell line are diploid. In some embodiments, cells of the cell line are engineered to produce a recombinant biological product and/or to enhance production of a non-recombinant biological product.

In one aspect, the present disclosure provides a composition comprising a Spodoptera frugiperda cell line selected from the group consisting of cell lines deposited as accession numbers PTA-127562, PTA-127563, PTA-127565, PTA-127566, PTA-127567, and PTA-127568. In some embodiments, the composition further comprises a preservative, cryoprotectant, solvent, buffer, media, antimicrobial agent, antibacterial agent, antifungal agent, or other agent.

In some embodiments, the cell line is free of Sf9 rhabdovirus. In some embodiments, the cell line is free of nucleic acid sequences having at least 70%, 75%, 80%, 85%, 90%, 95%, 98%, or 99% homology to the sequence set forth in NCBI Accession No. KF947078.

In some embodiments, the cell line is lyophilized. In some embodiments, cells of the cell line are diploid. In some embodiments, cells of the cell line are engineered to produce a recombinant biological product and/or to enhance production of a non-recombinant biological product.

In one aspect, the present disclosure provides a kit comprising one or more Spodoptera frugiperda cell lines deposited as accession number PTA-127562, PTA-127563, PTA-127565, PTA-127566, PTA-127567, or PTA-127568.

In some embodiments, the composition further comprises a preservative, cryoprotectant, solvent, buffer, media, antimicrobial agent, antibacterial agent, antifungal agent, or other agent. In some embodiments, cells of the cell line are suspended, stored, or maintained in a preservative, cryoprotectant, solvent, buffer, media, antimicrobial agent, antibacterial agent, antifungal agent, or other agent.

In some embodiments, the cell line is free of Sf9 rhabdovirus. In some embodiments, the cell line is free of nucleic acid sequences having at least 70%, 75%, 80%, 85%, 90%, 95%, 98%, or 99% homology to the sequence set forth in NCBI Accession No. KF947078.

In some embodiments, the cell line is lyophilized. In some embodiments, cells of the cell line are diploid. In some embodiments, cells of the cell line are engineered to produce a recombinant biological product and/or to enhance production of a non-recombinant biological product.

In one aspect, the present disclosure provides a method of making a diploid Sf9 cell line free of Sf9 rhabdovirus, the method comprising the steps of:
subjecting a suspension of Sf9 cells to segregation using an automated cell sorter to obtain one or more single-cell isolates;
maintaining the one or more single-cell isolates under conditions that promote cell growth and division to obtain one or more clonal cell cultures;
expanding the one or more clonal cell cultures under conditions that promote cell growth and division to obtain one or more clonal cell lines;
subjecting the one or more clonal cell lines to molecular testing to detect the presence or absence of Sf9 rhabdovirus; and
subjecting the one or more clonal cell lines to further molecular testing to detect the ploidy of the cell line.

In some embodiments, the automated cell sorter is a cell printer. In some embodiments, conditions that promote cell growth and division comprise the use of standard cell culture media. In some embodiments, conditions that promote cell growth and division comprise the use of conditioned cell culture media.

In some embodiments, the molecular testing comprises the detection of Sf rhabdovirus nucleic acid sequences. In some embodiments, the molecular testing comprises PCR amplification of Sf rhabdovirus nucleic acid sequences.

In some embodiments, the further molecular testing to detect the ploidy of the cell line comprises karyotyping. In some embodiments, the further molecular testing to detect the ploidy of the cell line comprises quantitative PCR.

In one aspect, the present disclosure provides a method of making a biological product free of Sf9 rhabdovirus, the method comprising the steps of culturing one or more Spodoptera frugiperda cell lines deposited as accession number PTA-127562, PTA-127563, PTA-127565, PTA-127566, PTA-127567, or PTA-127568 under conditions that promote production of the biological product, maintaining the culture for a time sufficient for the accumulation of biological product, and isolating the biological product.

In some embodiments, the biological product is a recombinant biological product or a non-recombinant biological product. In some embodiments, the biological product is an RNA, a DNA, a polysaccharide, a lipid, a peptide, a protein, an enzyme, an antibody, or a vaccine.

### BRIEF DESCRIPTION OF THE DRAWINGS

FIG. 1 is an image illustrating the isolation of individual Sf9 cells from a suspension of cells through the use of a cell printer.

FIG. 2A-G are images illustrating the development of a single-cell isolate to a clonal cell culture over a period of nine days. Images were taken on the following days/times: 0, 1 (1:23 hours), 2 (0:33 hours), 2 (2 17:35 hours), 5 (23:55 hours), 7 (20:35 hours), and 9 (17:41 hours).

### DETAILED DESCRIPTION

Provided herein are new Sf9 cell lines free of Sf9 rhabdovirus, methods for making and using the same, and kits comprising the same. In one aspect, the cell lines are useful for the production of biological products, including pharmaceuticals.

The following terms are used herein, the definitions of which are provided for guidance. Where not defined, terms used herein are given to have their accepted meaning in the art.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the present technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “Sf9 rhabdovirus” refers to the viral sequence set forth in NCBI Accession No. KF947078 and given herein as SEQ ID NO: 1 (Table 5).

As used herein, the term “clonal cell cultures” refer to a cell culture derived from a single cell isolate.

As used herein, the term “clonal cell line” refers to an established cell line derived from a clonal cell culture.

As used herein, the term “derived” or “derived from” refers to that having been obtained from the identified source, directly or indirectly. With respect to a cell, cell culture, or cell line, the reference identifies the original source material. For example, the reference Sf9-derived cell line refers to a cell line derived from a parent Sf9 cell line. The companion reference “subclone” refers to the material derived from the identified original source. For example, an Sf9 cell line derived from a parent Sf9 line may be referred to as a subclone of that line.

One of ordinary skill in the art will understand that “conditions that promote cell growth and division” refers to conditions suitable for growing a particular cell type. These are readily ascertainable from a variety of sources, for example but not limited to, cell culture manuals, commercial cell banks, or vendors of culture media and/or plastic ware, and may vary depending on environmental or other conditions. One of ordinary skill in the art will further understand that the conditions will periodically require optimization.

As used herein, “testing for the presence of Sf9 rhabdovirus” and related phrases mean detecting the presence of the virus by any means available. One of ordinary skill in the art will understand that there are a wide range of methods available for this, including but not limited to PCR-based methods (Reverse Transcription (RT), RT-Polymerase Chain Reaction (RT-PCR), RT-PCR coupled with nested PCR, quantitative PCR (real-time PCR)), probe hybridization techniques, DNA sequencing, electron microscopy, antibody-based detection (e.g., ELISA), and infection-based methods.

As used herein, references to testing the ploidy of cells, cell cultures, or cell lines means detecting the chromosomal complement by any means available. One of ordinary skill in the art will understand that there are a wide range of methods available for this including karyotyping metaphase chromosomes and methods for quantifying the DNA content of cells.

As used herein, “biological product” refers generally to a product, molecule, or material produced in a cell, cell culture, cell line, or organism. The term encompasses recombinant and non-recombinant products, whether or not the product is intended for use as a therapeutic. The term encompasses so-called end of production cell (EOPC) material, as well as production intermediates. As used herein, the biological product may be a natural product of the cell, cell culture, cell line, or organism, or may be produced by engineering or manipulation that causes production of the product.

In some embodiments, the biological product is a recombinant biological product or a non-recombinant biological product. In some embodiments, the biological product is an endogenous biological product. In some embodiments, the production of an endogenous biological product is enhanced. In some embodiments, the production of an endogenous product is enhanced by spontaneous or induced genetic changes, changes to culture conditions, or by the addition of a pharmacological agent.

In some embodiments, the biological product is an RNA or a DNA. In some embodiments, the biological product is a polysaccharide, lipid, or protein. In some embodiments, the biological product is an enzyme, antibody, or peptide. In some embodiments, the biological product is a vaccine.

In some embodiments, the biological product is a virus or virus like particle. In some embodiments, the biological product is a virus like particle containing antigen from a single virus strain. In some embodiments, the biological product is a virus like particle containing antigens from multiple virus strains.

In some embodiments, the biological product is useful for the treatment or prevention of a disease, or as an intermediate in the production of a therapeutic composition. In some embodiments, the biological product is useful for vaccination against a disease or in the treatment of an infection.

In some embodiments, the biological product is useful for a biological assay. In some embodiments, the biological product is useful for a diagnostic assay. In some embodiments, the biological product is useful in a growth medium. In some embodiments, the biological product is altered. In some embodiments, the biological product is altered by radiolabeling or glycosylation.

In some embodiments, the biological product is an end of production cell (EOPC) material. In some embodiments, the biological product is a production intermediate.

As used herein, “recombinant biological product” refers generally to a product, molecule, or material produced in a cell, cell culture, cell line, or organism that has been accordingly engineered, induced, or otherwise caused to produce the biological product. As used herein, the term refers in particular to products, molecules, or materials that are not endogenous to the cell, cell culture, cell line, or organism. The term encompasses products, molecules, or materials for which the cell, cell culture, cell line, or organism comprises an endogenous counterpart, wherein the cell, cell culture, cell line, or organism has been accordingly engineered to include one or more exogenous copies, versions, homologues, orthologues, etc., as well as non-natural versions of the product, such those having truncations, insertions, fusions, etc.

As used herein, “non-recombinant biological product” refers generally to a product, molecule, or material produced in a cell, cell culture, cell line, or organism from an endogenous source. The term encompasses products, molecules, or materials produced naturally by the cell, cell culture, cell line, or organism, whether produced constitutively or non-constitutively. The term includes products, molecules, or materials produced in a condition-specific manner, such as those produced under specific conditions of growth or maintenance. Such conditions include but are not limited to conditions of nutrient availability, cell density, temperature, oxygenation, physical agitation, light exposure, etc. The term includes products that have been mutated either spontaneously or deliberately to produce a non-wild-type product. In some embodiments, the non-wild-type product possesses reduced activity or increased activity compared to the wild-type product. In some embodiments, the non-wild-type product possesses a new activity as compared to the wild-type product, such as a gain-of-function activity. The term encompasses products, molecules, or materials for which the cell, cell culture, cell line, or organism comprises an endogenous counterpart, wherein the cell, cell culture, cell line, or organism has spontaneously developed or has been made to develop a mutation leading to expansion of cellular machinery for production of the product, or multimerization of a gene, component of a gene, gene region, chromosome, etc.

In one aspect, the present disclosure provides Spodoptera frugiperda cell lines deposited as accession numbers PTA-127562, PTA-127563, PTA-127565, PTA-127566, PTA-127567, and PTA-127568. In some embodiments, the cells are maintained, suspended, or otherwise directly contacted by a preservative, solvent, buffer, media, antimicrobial agent, antibacterial agent, antifungal agent, or other agent. In some embodiments, the preservative is a cryoprotectant. In some embodiments the preservative is a cryoprotectant selected from the group consisting of a nucleotide, a disaccharide, a polyol, and a polysaccharide. In some embodiments, the cryoprotectant is selected from the group consisting of DMSO, inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, and skim milk. In some embodiments, the cryoprotectant selected from the group consisting of DMSO, Trehalose, or a combination thereof.

The Spodoptera frugiperda cell lines described and claimed herein were deposited with an approved authority pursuant to the Budapest Treaty, as outlined below.

| Cell | Accession | |
|---|---|---|
| Line | Number | Deposited |
| 281G3 | PTA-127562 | May 5, 2023 |
| 282H10 | PTA-127563 | May 5, 2023 |
| 284C2 | PTA-127565 | May 5, 2023 |
| 281G3-2 | PTA-127566 | May 5, 2023 |
| 284C2-2 | PTA-127567 | May 5, 2023 |
| 281A5 | PTA-127568 | May 5, 2023 |

In one aspect, the present disclosure provides Spodoptera frugiperda cell lines that are free of the Sf9 rhabdovirus. In particular, they are free of nucleic acid sequences having at least 70%, 75%, 80%, 85%, 90%, 95%, 98%, or 99% homology to the sequence set forth in NCBI Accession No. KF947078. The cells may be maintained as live cultures or stored in any way suitable, such as in a lyophilized state.

In one aspect, the present disclosure provides Spodoptera frugiperda cell lines that are clonal. It has been reported that the ATCC CRL-1711 line is a heterogeneous population of cells, which is disfavored for the production of biological products. Sf9 lines described and claimed herein were derived from a single Sf9 cell, and therefore comprise homogeneous cell populations.

In one aspect, the present disclosure provides Spodoptera frugiperda cell lines that are diploid. Tetraploidy occurs when Sf9 cells are under stress, and is disfavored for the production of biological products. Sf9 lines described and claimed herein comprise homogeneous populations of diploid cells.

In one aspect, the present disclosure provides Spodoptera frugiperda cell lines that are engineered to produce a recombinant biological product.

In one aspect, the present disclosure provides Spodoptera frugiperda cell lines that produce increased levels of a non-recombinant biological product as compared to wild-type cells.

In one aspect, the present disclosure provides a composition comprising a Spodoptera frugiperda cell line selected from the group consisting of cell lines deposited as accession numbers PTA-127562, PTA-127563, PTA-127565, PTA-127566, PTA-127567, and PTA-127568. In some embodiments, the composition further comprises preservative, cryoprotectant, solvent, buffer, media, antimicrobial agent, antibacterial agent, antifungal agent, or other agent. As described herein, the cell lines disclosed and claimed herein are Sf9 rhabdovirus, and free of nucleic acid sequences having at least 70%, 75%, 80%, 85%, 90%, 95%, 98%, or 99% homology to the sequence set forth in NCBI Accession No. KF947078. The cells are diploid, may be used for the production of a biological product, and may be stored in any way suitable.

In one aspect, the present disclosure provides kits comprising one or more Spodoptera frugiperda cell lines selected from the group consisting of cell lines deposited as accession numbers PTA-127562, PTA-127563, PTA-127565, PTA-127566, PTA-127567, and PTA-127568. In some embodiments, the kits further comprise a preservative, cryopreservative, solvent, buffer, media, antimicrobial agent, antibacterial agent, antifungal agent, or other agent. In some embodiments, the cell line is maintained, suspended, or otherwise directly contacted by the preservative, cryopreservative, or other agent. As described herein, the cell lines are diploid and free of Sf9 rhabdovirus. In some embodiments, the cell lines of the kits are engineered to produce a recombinant biological product. In some embodiments, the cell lines of the kits produce a non-recombinant biological product.

In some embodiments, the kit comprises a package insert with instructions on optimal growth conditions in monolayer or suspension culture. In some embodiments, the kit comprises reagents for maintaining and/or expanding the Sf9 cells.

In some embodiments, the kit comprises a cell culture media. In some embodiments, the kit comprises ESF 921, a chemically defined media, ExpiSf CD, Sf900 II, Sf900 III, Express Five SFM, Bac Vector, InsectaGro, SFM4Insect, SFX-Insect, IPL41, IS Sf Insect, Schneider's, TriEx, Insect-EXPRESS, TheraPEAK, or any combination thereof. In some embodiments, the kit comprises ESF-AF media. In some embodiments the kit comprises flasks for cell growth. In some embodiments, the kit comprises one or more sets of forward and reverse primers that can be used in a PCR to confirm the absence of Sf9 rhabdovirus over time.

In some embodiments, the kit comprises a probe sequence. In some embodiments, the probe sequence is labeled with a fluorophore. In some embodiments, the kit comprises an enzyme. In some embodiments, the kit comprises a chaotropic agent. In some embodiments, the kit comprises a cell line permissive of Sf9 rhabdovirus replication for use in infection-based methods. In some embodiments, the kit comprises a Spodoptera exigua cell line.

In one aspect, the present disclosure provides methods for making a diploid Sf9 cell line free of Sf9 rhabdovirus. In some embodiments, the method comprises the steps of subjecting a suspension of Sf9 cells to segregation using an automated cell sorter to obtain one or more single-cell isolates; maintaining the one or more single-cell isolates under conditions that promote cell growth and division to obtain one or more clonal cell cultures; expanding the one or more clonal cell cultures under conditions that promote cell growth and division to obtain one or more clonal cell lines; subjecting the one or more clonal cell lines to molecular testing to detect the presence or absence of Sf9 rhabdovirus; and subjecting the one or more clonal cell lines to further molecular testing to detect the ploidy of the cell line.

One of ordinary skill in the art will understand that the step of subjecting a suspension of Sf9 cells to segregation using an automated cell sorter to obtain one or more single-cell isolates may be accomplished by a variety of means. In some embodiments, the automated cell sorter is a flow cytometer. In some embodiments, the automated cell sorter is a cell printer.

In some embodiments, “conditions that promote cell growth and division” comprise the use of standard cell culture media. In some embodiments, conditions that promote cell growth and division comprise the use of conditioned cell culture media. One of ordinary skill in the art will understand that conditions must be directed to growing a particular cell type, and are readily ascertainable from a variety of sources. One of ordinary skill in the art will further understand that the conditions will initially and/or periodically require optimization.

In some embodiments, molecular testing for the Sf9 rhabdovirus comprises the detection of viral nucleic acid sequences by PCR. However, one of ordinary skill in the art will recognize that a wide range of methods may be used, including PCR-based methods, probe hybridization techniques, DNA sequencing, electron microscopy, antibody-based detection (e.g., ELISA), and infection-based methods. For demonstrations included herein, Sf9 rhabdovirus was detected using PCR-based methods with direct sequencing of select amplification products. It is to be understood that the claims are not limited to these illustrative methods.

In some embodiments, cell lines disclosed and claimed herein are subject to further molecular testing to determine the ploidy of the cells. One of ordinary skill in the art will recognize that any suitable method may be used for this step, such as karyotyping metaphase chromosomes or methods for quantifying the DNA content of cells. For demonstrations included herein, ploidy was determined by karyotyping metaphase chromosomes. It is to be understood that the claims are not limited to these illustrative methods.

In one aspect, the present disclosure provides methods for making a biological product free of Sf9 rhabdovirus. In some embodiments, the method comprises the steps of culturing one or more Spodoptera frugiperda cell lines deposited as accession number PTA-127562, PTA-127563, PTA-127565, PTA-127566, PTA-127567, or PTA-127568 under conditions that promote production of the biological product, maintaining the culture for a time sufficient for the accumulation of biological product, and isolating the biological product by means appropriate to the biological product. For example, appropriate means to isolate biological products can include, but is not limited to, lipid isolation and purification methods, protein isolation and purification methods, polysaccharide isolation and purification methods, and virus or virus like particle isolation and purification methods. Appropriate means to isolate the biological product will be known by one with skill in the art, who will further understand that routine optimization will result in enhanced yields.

In some embodiments, the biological product is a recombinant biological product or a non-recombinant biological product. In some embodiments, the biological product is an RNA, a DNA, a polysaccharide, a lipid, a peptide, a protein, an enzyme, an antibody, or a vaccine.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Isolation and Expansion of Sf9 Rhabdovirus-Free Sf9 Cell Lines

This example demonstrates methods and compositions for making clonal, Sf9 rhabdovirus-free Sf9 cell lines.

Clonal Sf9 cell lines were established by subjecting a suspension of Sf9 cells to segregation using an automated cell sorter to obtain one or more single-cell isolates, culturing the single-cell isolates under conditions that promote cell growth and division to obtain one or more clonal cell cultures; expanding the one or more clonal cell cultures under conditions that promote cell growth and division to obtain one or more clonal cell lines; and subjecting the one or more clonal cell lines to molecular testing to detect the presence or absence of Sf9 rhabdovirus.

### Isolation and Outgrowth of Sf9 Cell Lines

Individual Sf9 cells, traceable to ATCC CRL-1711 lot number 70020337 (passage 10), were printed into the wells of a 96-well plate (Corning Cellbind 3300 series) using a CloneSelect Single Cell f sight printer. The manufacturer standard protocols for single cell printing were used with cell parameters set to insect cell size (Operation, Maintenance, and Calibration of the f.sight CloneSelect Single Cell Printer (Doc ID 302431), Operation, Maintenance, and Calibration of the CloneSelect Imager (Doc ID 302422), CEDEX Cell Analyzer Operation and Maintenance (Doc ID 15710), Vi-Cell XR Cell Viability Analyzer Operation and Maintenance (Doc ID 239033), Operation and Maintenance of the Vi-Cell BLU Cell Viability Analyzer (Doc ID 758926), and Insect Cell Culture Procedure (Doc ID 29844)).

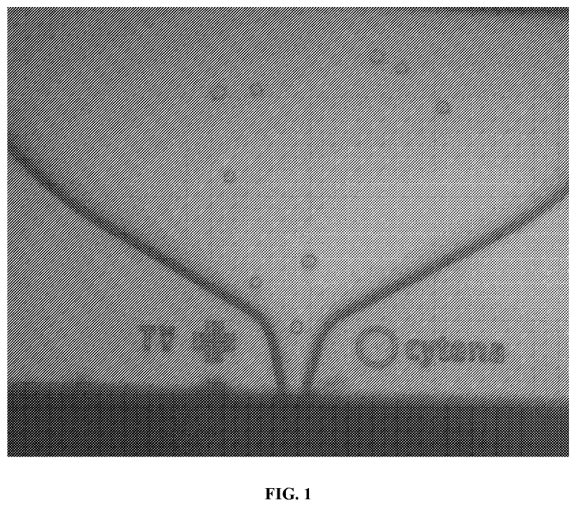

A single-use cartridge was used to print into each well to prevent cross-contamination between cell lines. A total of five 96-well plates were established, with cells printed into either standard media (90% Grace's Media and 10% FBS) or insect cell conditioned media (45% Grace's Media (Gibco 11605-094), 45% Conditioned Grace's Media; 10% FBS (Sigma F4135)). Conditioned media was generated using the supernatant of replicating Trichoplusia ni cells according to routine methods known in the art. Cells were imaged as they were printed to confirm the deposition of single isolated cells, as shown in FIG. 1.

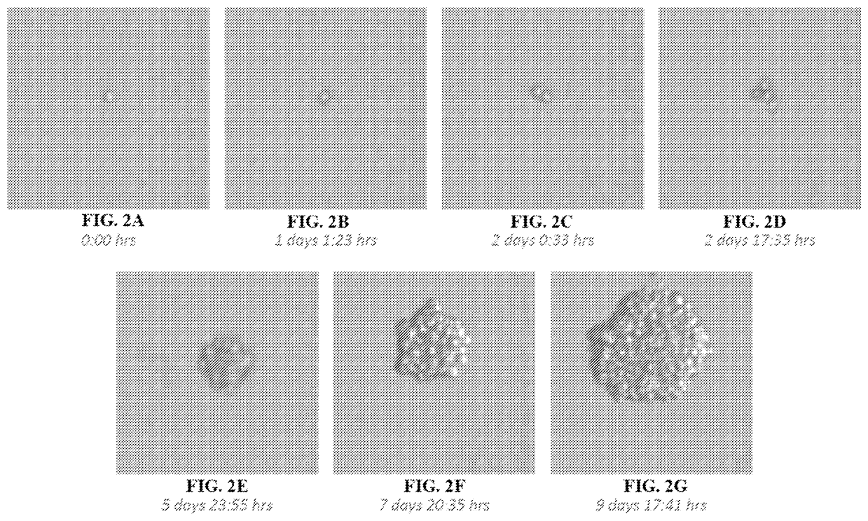

Cells were allowed to settle for 2 hours before being imaged using a CloneSelect Imager to identify the day 0 state and confirm single cell cultures. An illustrative image is shown in FIG. 2A. Plates were imaged regularly throughout expansion to track colony growth. FIG. 2A-2G. During the first 14 days, 50% of the media in each was replaced weekly using either standard media or insect cell conditioned media as per the initial culture conditions. At day 14 post printing, all cells were switched to standard media.

Colonies at 20-45% confluence were transferred to 24-well plates in 1.0 ml of standard media. Cells were passaged by using percussive force to break adherence, and cell number was determined using an automated cell counter and cells were passaged at the desired density. At 15%-40% confluence cells were transferred to T25 flasks at a density of 1×10⁶ cells per flask and grown to between 50%-80% confluence, with fresh media at day 3 post passage if cells were below 50% confluence. During T25 outgrowth a cell sample was removed for Sf9 rhabdovirus testing, as described below.

Sf9 rhabdovirus-free cell lines at 50%-80% confluence were then passaged into T75 flasks at a density of 3×10⁶ cells per flask. T75 flasks at 50-80% confluence were passaged into a 50 ml suspension culture and grown in an Erlenmeyer flask using Grace's media supplemented with 10% FBS and 0.1% Pluronic (Kolliphor P-188). All cell lines were then adapted to grow in ESF-AF media (ESF-AF 99-300) in a stepwise manner, by first passaging cells in ESF-AF supplemented with 10% FBS and then passaging cells with gradual reductions in FBS supplementation until growth was achieved in unsupplemented ESF-AF. Cells were then seeded into T150 flasks at a density of 6×10⁶ cells per flask for continued passaging, with all Sf9 rhabdovirus-free cell lines exceeding passage 90 at the time of filing.

### Sf9 Rhabdovirus Detection Protocol

Cell samples were removed from cell lines at the T25 and T75 outgrowth stages to test for the presence of Sf9 rhabdovirus via PCR and sequencing. As shown in Table 1, a total of 5 Sf9 rhabdovirus-free cell lines were isolated.

TABLE 1
Sf9 Rhabdovirus Testing Results Summary

| | Conditioned | | # Colonies | # Colonies | # Colonies | |
|---|---|---|---|---|---|---|
| | media/ | # | Transferred | Sf9 | Sf9 | % |
| | Unconditioned | Colonies | to 24-well | rhabdovirus- | rhabdovirus- | Rhabdovirus- |
| Plate | media | Obtained | plate | positive | negative | negative |
| 281 | Conditioned | 32 | 17 | 14* | 2* | 11.76% |
| 282 | | 26 | 14 | 13 | 1 | 7.14% |
| 283 | | 10 | 5 | 4 | 1 | 20.00% |
| 284 | Unconditioned | 15 | 9 | 5\hat{ } | 1\hat{ } | 11.11% |
| 285 | | 1 | 1 | 0 | 0 | 0.00% |

*one colony discarded due to general contamination
\hat{ }three colonies discarded due to general contamination

Cell lines were deemed Sf9 rhabdovirus negative based on a negative result with PCRs for the Sf9 rhabdovirus N-gene and L-gene.

Nested PCRs were performed to test for the presence of the Sf9 rhabdovirus L-gene and the N-gene using the primer pairs in Table 2. Briefly, 5×10⁶ Sf9 cells were pelleted via centrifugation and total RNA was extracted using a Qiagen RNeasy Mini Kit (Product Number 74104) according to the manufacturer protocol. DNA was digested by treating samples with DNaseI (NEB M0303S) in DNase-I buffer (NEB B0303S) at 37° C. for 10 minutes, followed by a 10 minute heat shock at 75° C. Total RNA was then converted to cDNA using a NEB Protoscript II First Strand cDNA Synthesis kit (NEB E6560S) according to the manufacturer protocol.

TABLE 2
Sf9-Rhabdovirus-Specific Primers

| | | | | Product |
|---|---|---|---|---|
| | | | Nucleotide | Size |
| Primer | Target | Sequence (5′→3′) | Position | (bp) |
| Mono-1* | L | ggcaaggctgtttggattactgacc | 8365-8389 | 792 |
| Mono-2* | | acaggtttgcagctaaggaggaca | 9158-9135 | |
| Mono-1i* | | atatgagagccccagacacacagcc | 8571-8595 | 500 |
| Mono-2i* | | acgatgtggtgagagaaacactcct | 9071-9046 | |
| Mono-3* | | tggcgagggactgcttacagaagg | 10630-10653 | 730 |
| Mono-4* | | cacagccgggggtgcaatca | 11359-11340 | |
| Mono-3i | | cctattcaacagcctatcagattgt | 10745-10773 | 480 |
| | | tacg | | |
| Mono-4i | | ttgtgggctcatagctctaagaa | 11202-11224 | |
| Mono-5* | | acaggagatgcggaagacccctc | 12137-12159 | 826 |
| Mono-6* | | atctcgcaggtgggacaacccc | 12962-12941 | |
| Mono-5i | | attgtgaattccgttcatggtatct | 12218-12242 | 474 |
| Mono-6i | | agatgagtagataggtctgagatcc | 12667-12691 | |
| Mono-7# | N | gaacttcattgctctaacaatgctt | 842-866 | 296 |
| Mono-8# | | aagtggtgtgggtgagaagg | 1118-1137 | |
| Mono-7i# | | tgcctttctgaaggagcaat | 902-921 | 192 |
| Mono-8i# | | gaagggtttctccgttcctc | 1074-1093 | |

*primer pairs validated in Ma H, Galvin TA, Glasner DR, Shaheduzzaman S, Khan AS.
Identification of a novel rhabdovirus in Spodoptera frugiperda cell lines. J Virol. 2014; 88:6576-6585.
#primer pairs validated in Rhabdovirus status test for Sf-RVNTM cell line using a nested PCR approach, Millipore (2020).

Total cDNA was then assayed via a series of nested PCRs. The first PCR was performed using the outer primer pairs, Mono-1/2, Mono-3/4, Mono-5/6, and Mono-7/8. The second PCR was performed on the product of the first PCR using the corresponding inner primer pairs Mono-1i/2i, Mono-3i/4i, Mono-5i/6i, and Mono-7i/8i, respectively. All PCRs were performed using Taq DNA polymerase (NEB M0273L), Taq Buffer (NEB B9014S), and 100 mM dNTPs (Agilent 200145-51), using cycling conditions recited in Table 3.

TABLE 3
PCR Conditions

| | Tempera- | | | |
|---|---|---|---|---|
| PCR Primers | ture (° C.) | Time | | Cycles |
| Mono-1/2, | 94 | 3 | min | 1 |
| Mono-3/4, | 94 | 30 | sec | 35 |
| Mono-5/6, and | 55 | 1 | min | |
| Mono-1i/2i | 72 | 1 | min | |
| | 72 | 10 | min | 1 |
| Mono-3i/4i and | 94 | 3 | min | 1 |
| Mono-5i/6i | 94 | 30 | sec | 35 |
| | 50 | 1 | min | |
| | 72 | 1 | min | |
| | 72 | 10 | min | 1 |
| Mono-7/8 and | 98 | 2 | min | 1 |
| Mono-7i/8i | 98 | 5 | sec | 30 |
| | 55 | 5 | sec | |
| | 72 | 15 | sec | |
| | 72 | 1 | min | 1 |

PCR products were analyzed by standard gel electrophoresis as compared to a 1 Kb DNA ladder (Invitrogen 10787018). A total of 5 clones were identified as negative for the N-gene and for L-gene primer sets Mono-3/4 and Mono-5/6, but positive for L-gene primer sets Mono-1/2 (281A5, 281G3, 282H10, and 284C2). Two clones (283B11 and 284A8) were identified as positive for rhabdoviral genes. Further testing was performed to determine if the Mono-1/2 PCR product from each clone was the result of an endogenous viral element (EVE) originating from the rhabdoviral L-gene, or a full copy of the wild type Sf9.

As shown below in Table 4, the Monoli/2i primers can bind non-specifically to sites on L gene-derived EVEs that are highly similar in sequence to the wild type L-gene binding sites. Geisler, et al.

TABLE 4
Non-specific binding of Mono-1i/2i
to L-gene and EVE-L1 sequences

| | Mono 1i | Mono 2i |
|---|---|---|
| | mismatches | mismatches |
| | shown in | shown in |
| | bold underline | bold underline |
| Primer | ATATGAGAGCCCC | ACGATGTGGTGAG |
| | AGACACACAGCC | AGAAACAC-TCCT |
| L Gene | ATATGAGAGCCCC | ACGATGTGGTGAG |
| | AGACACACAGCC | AGAAACACCTCCT |
| EVE-L1 | ACATGAGAGCCCC | AGGATGTGGTAAG |
| | CGATACCCAGCC | TGAAACTCCTCCT |

For clones 281A5, 281G3, 282H10, 283B11, 284A8, and 284C2, the Mono-1/2 and Mono-1i/2i PCR product was directly sequenced to determine whether it derived from EVE-L1 or the wild type L gene. Briefly, total RNA was extracted from 5×10⁶ cells from each clone and cDNA was generated as previously described. Templates for Sanger sequencing were generated using the Mono-1/2 and Mono-1i/2i PCRs as previously described, and sequencing was performed. The resulting Sanger sequences were aligned with the published Sf9 genome to determine the percent identity to both the EVE-L1 sequence and the wild type L-gene sequence.

Clones 281A5, 281G3, 282H10, and 284C2 all showed above 98% sequence identity to the EVE-L1 sequence (98.88%, 99.55%, 99.77%, 98.86%, and 99.77% identity, respectively). Conversely, clones 283B11 and 284A8 showed 99.3% and 100% sequence identity to the wild-type viral L-gene sequence, respectively. Accordingly, clones 283B11 and 284A8 were discarded as virally infected, and clones 281A5, 281G3, 282H10, and 284C2 were identified as virus-free and containing the EVE-L1 sequence.

To confirm this result, a single clone was subjected to an additional round of Illumina sequencing. Briefly, the Mono-1i/2i PCR product from clone 281A5 was cleaned using a Qiagen PCR and Gel Cleanup Kit (28706X4) per the manufacturer protocol, and the resulting DNA was quantified using a DeNovix dsDNA Broad Range Kit (DSDNA-BROAD-EVAL) per the manufacturer protocol. DNA was sequenced on an Illumina sequencer using the manufacturer protocol, and the resulting sequence data was analyzed using Geneious Prime. Sequence alignment analysis confirmed that the amplification product in the Mono-1i/2i PCR was the EVE-L1 and not the wild type L-gene.

These results show that methods and compositions disclosed herein are useful for the isolation of clonal Sf9 cell lines that are free of Sf9 rhabdovirus. Accordingly, methods and compositions of the present disclosure are useful and effective for the production of cell lines and their subsequent use for the production of recombinant biological products.

### Example 2: Rhabdovirus-free Sf9 Cell Lines are Diploid

This example will demonstrate that the Sf9 rhabdovirus-free Sf9 cell lines of the present disclosure are diploid.

Sf9 cells are known to exist in diploid or tetraploid states. It is predicted that the clonal cell lines will be diploid based on the size of the cells under microscopic observation. The ploidy of the Sf9 rhabdovirus-free cell lines 281A5, 281G3, 282H10, and 284C2 will be determined using standard methods in the art.

The results are predicted to confirm that the 281A5, 281G3, 282H10, and 284C2 cell lines are diploid. Accordingly, the Sf9 rhabdovirus-free Sf9 cell lines of the present disclosure are useful in methods and compositions that use such cells, such as for the production of recombinant biological products.

### Example 3: Methods for the Production of Biological Products

This example will demonstrate use of the Sf9 rhabdovirus-free Sf9 cell lines of the present disclosure for the production of biological products.

Sf9 cells of the present disclosure will be cultured under conditions that promote production of the biological product. One of ordinary skill in the art will understand that such conditions must be suitable for the particular product to be produced, which are readily ascertainable from a variety of sources, for example but not limited to, cell culture manuals, commercial cell banks, or vendors of culture media and/or plastic ware, and may vary depending on environmental or other conditions. One of ordinary skill in the art will further understand that the conditions will initially and/or periodically require optimization.

In some embodiments, the biological product is a recombinant biological product or a non-recombinant biological product. In some embodiments, the biological product is an endogenous biological product. In some embodiments, the production of an endogenous biological product is enhanced. In some embodiments, the production of an endogenous product is enhanced by spontaneous or induced genetic changes, changes to culture conditions, or by the addition of a pharmacological agent.

In some embodiments, the biological product is an RNA or a DNA. In some embodiments, the biological product is a polysaccharide, lipid, or protein. In some embodiments, the biological product is an enzyme, antibody, or peptide. In some embodiments, the biological product is a vaccine.

In some embodiments, the biological product is a virus or virus like particle. In some embodiments, the biological product is a virus like particle containing antigen from a single virus strain. In some embodiments, the biological product is a virus like particle containing antigens from multiple virus strains.

In some embodiments, the biological product is useful for the treatment or prevention of a disease, or as an intermediate in the production of a therapeutic composition. In some embodiments, the biological product is useful for vaccination against a disease or in the treatment of an infection.

In some embodiments, the biological product is useful for a biological assay. In some embodiments, the biological product is useful for a diagnostic assay. In some embodiments, the biological product is useful in a growth medium. In some embodiments, the biological product is altered. In some embodiments, the biological product is altered by radiolabeling or glycosylation.

In some embodiments, the biological product is an end of production cell (EOPC) material. In some embodiments, the biological product is a production intermediate.

Following culture of the Sf9 cells of the present disclosure for a suitable time period and under suitable conditions for production of the biological product, the product will be harvested and or isolated using methods suitable for the particular product.

These results will show that the cell lines of the present disclosure are able to produce a wide array of biological products for a variety of purposes and uses. Accordingly, these results will demonstrate that the Sf9 rhabdovirus-free Sf9 cell lines of the present disclosure are useful for the production of biological products.

### Example 4: Kits Comprising Rhabdovirus-free Sf9 Cell Lines

This example demonstrates incorporation of the Sf9 rhabdovirus-free Sf9 cell lines of the present disclosure into kits suitable for various purposes. Sf9 cells are a commonly used eukaryotic system for the production of biological products. Accordingly, kits comprising Sf9 cells of the present disclosure are of general value.

In some embodiments, the kit comprises one or more of the Sf9 rhabdovirus-free Sf9 cell lines of the present disclosure.

In some embodiments, the kit comprises a preservative. In some embodiments, the kit comprises a cryoprotectant. In some embodiments, the Sf9 cells of the kit are suspended in, maintained in, or otherwise in direct contact with the preservative or cryoprotectant.

In some embodiments, the kit comprises a package insert with instructions on optimal growth conditions in monolayer or suspension culture. In some embodiments, the kit comprises reagents for maintaining and/or expanding the Sf9 cells.

In some embodiments, the kit comprises a cell culture media. In some embodiments, the kit comprises ESF-AF media. In some embodiments the kit comprises flasks for cell growth. In some embodiments, the kit comprises one or more sets of forward and reverse primers that can be used in a PCR to confirm the absence of Sf9 rhabdovirus over time.

In some embodiments, the kit comprises a probe sequence. In some embodiments, the probe sequence is labeled with a fluorophore. In some embodiments, the kit comprises an enzyme. In some embodiments, the kit comprises a chaotropic agent. In some embodiments, the kit comprises a cell line permissive of Sf9 rhabdovirus replication for use in infection-based methods. In some embodiments, the kit comprises a S. exigua cell line.

In some embodiments, aspects of the kit are used to show that a Sf9 cell line is free of Sf9 rhabdovirus contamination. In some embodiments, aspects of the kit are used to produce a biological product.

These results will show that the kits of the present disclosure can be used to produce biological products in Sf9 cells and to determine whether a cell line is free of Sf9 rhabdovirus infection. Accordingly, these results will demonstrate that the kits of the present disclosure are useful for the production of biological products and the assaying of cell lines for Sf9 rhabdovirus.

### Example 5: Demonstration of Sf9 Rhabdovirus-Free Cell Lines by qPCR

This example will demonstrate that the Sf9 rhabdovirus-free Sf9 cell lines of the present disclosure are not infected with Sf9 rhabdovirus using qPCR methodology.

The Sf9 rhabdovirus genome is actively expressed during viral replication in infected Sf9 cells, creating transcripts from the wild-type N-, P-, G-, and L-genes, whereas the corresponding N-, P-, G-, and L-EVE's do not create equivalent transcripts in uninfected Sf9 cells. The infection status of the Sf9 rhabdovirus-free cell lines 281A5, 281G3, 282H10, and 284C2 will be determined using standard qPCR methods in the art to assay for N-, P-, G-, and L-gene transcripts.

The results are predicted to show that the 281A5, 281G3, 282H10, and 284C2 cell lines are Sf9-rhabdovirus free and do not express N-, P-, G-, or L-gene transcripts. Accordingly, these results will demonstrate that the Sf9 rhabdovirus-free Sf9 cell lines of the present disclosure are useful in methods and compositions that use such cells, such as for the production of recombinant biological products.

### Example 6: Demonstration of Sf9 Rhabdovirus-Free Cell Lines by Infectivity Assay

This example will demonstrate that the Sf9 rhabdovirus-free Sf9 cell lines of the present disclosure are not infected with Sf9 rhabdovirus using infectivity assay methodology.

Sf9 cells that are infected with Sf9 rhabdovirus release virions into media supernatant, whereas uninfected Sf9 cells do not release virions. The infection status of the Sf9 rhabdovirus-free cell lines 281A5, 281G3, 282H10, and 284C2 will be determined by performing an infectivity assay. Briefly, samples from a Sf9 rhabdovirus-free cell line will be treated with supernatant from multiple other cells lines, including a Sf9 rhabdovirus-infected Sf9 cell line, 281A5, 281G3, 282H10, and 284C2. Then the Sf9 rhabdovirus-free cell line samples will be assayed for Sf9 rhabdovirus infection using methods that are standard in the art.

The results are predicted to confirm that the 281A5, 281G3, 282H10, and 284C2 cell lines are Sf9-rhabdovirus free and do not release virions into media supernatant. Accordingly, these results will demonstrate that the Sf9 rhabdovirus-free Sf9 cell lines of the present disclosure are useful in methods and compositions that use such cells, such as for the production of recombinant biological products.

### Example 7: Alternative Protocol for the Isolation and Expansion of Sf9 Rhabdovirus-Free Sf9 Cell Lines

This example will demonstrate that additional Sf9 rhabdovirus-free Sf9 cell lines can be cloned and expanded using alternative protocols.

Clonal Sf9 cell lines exhibit variations in important cell culture attributes, such as growth rate, media tolerance, and protein expression levels. Thus, it is advantageous to continually adjust cloning methodologies to identify optimal clonal cell line populations that are Sf9 rhabdovirus free. Briefly, a heterogenic population of infected and virus-free Sf9 cells will be adapted to suspension culture in standard media (90% Grace's Media supplemented with 10% FBS). The Sf9 cells will then be adapted to grow in a chemically defined (CD) media, which will comprise a mixture of organic and inorganic elements. The CD media-adapted Sf9 cells will then be sorted into individual clonal cell populations in a media that is 50% CD media and 50% standard media. Clonal cell populations will be expanded using 100% CD media and individual clones will be assayed for Sf9 rhabdovirus infection using methods that are standard in the art.

The results are predicted to show that new Sf9 rhabdovirus-free Sf9 clonal cell lines have been established via isolation using the CD media, and that the clonal cell lines will possess variable cell culture attributes. Accordingly, these results will demonstrate that the Sf9 rhabdovirus-free Sf9 cell lines of the present disclosure are useful in methods and compositions that use such cells, such as for the production of recombinant biological products.

TABLE 5
SEQ ID NO: 1.
NCBI Accession No. KF947078

| | |
|---|---|
| 1 | tgaaaattat acgataaatg atcctaactc ttatagatga caacgactac ttcacggata |
| 61 | tcggcttctt agatcttatc atgacacagg gaaccatgaa gccagtatgg gaagaattgg |
| 121 | ggacaggaga aacagagttc caagggaccg tggacattcc agggagatct ctcaagccag |
| 181 | aaaaaacaga ttggagtgtt gatacatgtc gggagatcag tttaaatctg aagttacctg |
| 241 | gtgaaatatg gcaactggcc catcaagaaa ccatcttcaa cagatttctt acattttacg |
| 301 | ctactgggta tgttccaaat acacacacag ccacagaaat tgtactctcc atggcatcac |
| 361 | taatcttcaa ggacaaggcc aaagcaccta ttgatttgat ttgggatgac tcatttcaag |
| 421 | ctagtccctc tgaggagtgt gggttctccg ttgttggaga aactccattg gttatcggac |
| 481 | aacacccgga tgatgatgac tacacattga gagaagatga agaatcagcc gctatgaatg |
| 541 | aggaagaaaa aatacaagca gctctaaaaa ctttgggaat tcaagatact ccagtagacc |
| 601 | tgaaggatgc atctggaatt gtctttgaga caaaggagga cagagaacaa aggatcaaga |
| 661 | atgagaaagc tctacatgta gaggatgata tcaacgctct aactcagatt acaaaacaat |
| 721 | tcttgtttga gtattccaca ggctccctac agaaatttgt tgcaaaggct actactattt |
| 781 | tcatagataa taatgctact aacggcttca cccgtttgca tctccatgcc atcagagtca |
| 841 | tgaacttcat tgctctaaca atgcttagaa aggtaaccaa gtcaaatgcc cagatgatca |
| 901 | atgcctttct gaaggagcaa tacaagagaa atattgcctc cctaatcccc ggcgccctct |
| 961 | cctctgattt tgctcctccc agtaagagct gcattgataa actgacagct atttctaaga |
| 1021 | atgacccggc agtcagttca ttctttgcaa aggttgtgat gctcaacatg gaggaggaac |
| 1081 | ggagaaaccc ttctctggtt gcttgtcttg gggcttccct tctcacccac accacttgga |
| 1141 | atggaatggg gattttacat gttatttttg aagtttgtct attccatcag attagctgga |
| 1201 | agaggttggt cacagagtcc ctgacctcac taacaaagat gtcatggggt gaagtcagtc |
| 1261 | aattcctcat caagtatcaa gcaaagggaa atcctgaccc aacggttgcc tgggccagaa |
| 1321 | tcattgatga ttcttacttt atgagattaa ccatagtaaa tcatcccaca cttgctgcat |
| 1381 | tattagtgga atccctcata agatctcaga aagatgatgg aatcctgaat gccaactggg |
| 1441 | ccatccaaca cagggacacc atcaattatt atagggacgc tgccaagctt ctcactgata |
| 1501 | agctcacagg acagactgct acagtccaag cccttaccaa tgaagccgct gatctagtta |
| 1561 | gaacaatgaa tgcaggaccc tctagatacc acccaaggcc tagtaccctt atccccatgg |
| 1621 | tagatctaaa cccggaagac ttataagact tacctattat cccaagacta atttccataa |
| 1681 | taatccccaa aaagacaatt actgttattt tctattaaaa aaccaatgaa aattatgcag |
| 1741 | agaatattga gacatatagt atccttcttc tcaaagtcct ggtgccaatc ctccgatccc |
| 1801 | gctctagtgt gcgactgtga gtatcctcct ctcaagagaa actatcaact gatttactct |
| 1861 | ataatggctt cccactctct tgacaccatt gatctatctg aaattggatt gacaagggag |
| 1921 | gttctgactg gggttggcga ttacatgact ggacaaagac cggttccagc cttcaatcct |
| 1981 | ccagaggtcg gtcactcccc ctctgatgaa gtggcaaaac gattgggaga actgaagaat |
| 2041 | tactggactc agttagagga tcctcttgat gagagaattc tcaatacctt gaaagcgatc |
| 2101 | agcatcctga gcggagacac cagaggagat ctgagtggaa aatataaaca tctagtccgc |
| 2161 | attagcggag atgacatgcc ccaattattg gacgaactta tagacatctg tcttctgggg |
| 2221 | cctaagactc taattgctac cttacgaatg gcgataaccg cctataccgc tgcattagcc |
| 2281 | agaaatgcca agtccaccat ctcagatatt actaccgcat cagcagattt gatggtcatc |
| 2341 | actcagatga tacagtccca gcaggaatct ttccaatcat cattagagca tctctctcat |
| 2401 | gcttggaata acgtcgccag tggtatgact gcttatacag cggaactgga taagagaacc |
| 2461 | ctcaagttga cacagcttac accgccagtc aagcctgacc atcaccgggc cccctctaca |
| 2521 | gcctccagtc atacccctga tgcttcagtg gggcttcata taaacataac ccccggttgt |
| 2581 | gcatataagt cccaatttgg ggtattgact tgctgtccca atgggaacat tggttttctt |
| 2641 | gcaaataact ccgatggcac tgtgatagcc aaacttgtgg aagtaattag gaagccacgc |
| 2701 | cctctcacga cggccctcaa taagaacctg tctgagctga tcaattatgt caaggcaaat |
| 2761 | ccgaagatcc ttgctactta ctctacctct tctcctcagg ataagcttaa tatcctgaat |
| 2821 | gagatccact tctgcatccc agatcttacc aatcaatggg tcaaggcctg ataacattcc |
| 2881 | cttccctcac aaactctaaa gatatctcca tccttgatga catttttatt aattatattt |
| 2941 | aagtacgatt taagatctac ataccaaaac caaccatttt gatgattaac atgttattat |
| 3001 | aaaaaaccaa tgaaacttat acgttaattg agataagttt gatttcattg ttccttgtgt |
| 3061 | catcaccgaa ctttttgagc tcgaacaatg agtgctcttg aacggattgc acggagccta |
| 3121 | tcattgaaga agttgaaccc taggagaact ccaaagactc agcctattcc tgaaaaggct |
| 3181 | actgtctatc atcctttcat gctctcttat gatctcaatt tagctattga gggtaaaatc |
| 3241 | cacatctccg ctatcaccat tatagtgaat gccctttcct tagcttgggc aatagaactc |
| 3301 | tttcactctg actcatcgtg gtcagggtgt cttgagtact tttggaaatc catcaaggat |
| 3361 | aacatattgg catccataaa ccctcgagtt gatccaaatg gaacttgtca tatgatgaca |
| 3421 | tcaatcataa ctttcctcgg attctcagat ggatcctgca tcaactcaga agcagagcca |
| 3481 | agacagctca caggatctag atcctgggag atcatgtctc ctaatcagaa tctcattgtg |
| 3541 | ataaccctag gattcaaaat aaccttgaaa accttcgcac agcaccagag atacagcttg |
| 3601 | cgtgaccatg gattccacaa attggagatg ctcaacgaga aagagaagaa aatgttgaac |
| 3661 | tatatggggg tcaaacaatt aaaaccccag tatacacatg aaaagacatt cgagaaactc |
| 3721 | attctcaaga acaaaggtcc aaaggggtct cgtgtcaggg caattcttca ctctcaaagt |
| 3781 | cgtgacatgt ggtctccaac cgctccttct cctccaccca catatgaaga tggatcctca |
| 3841 | gatgaatggg atcagcaaca actgcacagc ctcaaccacc tgcatacacc ttctgtcccc |
| 3901 | ctgagggccc ccaggacatc cccaccccaa caactctccc caaaaccgac atccacaacc |
| 3961 | caacccctcc cacaactcac acaaccaaac aagccccaag aactctccaa gtagacactt |
| 4021 | gacagccccc accaattact tttagatcat aaaaaaccaa caggcagaat ataagaccta |
| 4081 | tcaattagag atattaaaaa atactaatta aacaattata catcaagcat tggctcatta |
| 4141 | tggttttctt aagtttatca acgatcatat ttatcctaag cctccgggct gtaacctgct |
| 4201 | ccaatcctct ctcctatcct aatggcattt tgactaacaa ctctactcac aatcatcccc |
| 4261 | tatcggactt ttatattttt tatgagaaca gttcccttac ctatactcaa ttccctgtgg |
| 4321 | ccccagactg ctctagtatt ctagatacta gagatgagca gtatcccacc actgttactt |
| 4381 | tgtggaaggt tgatcaagaa tctcaagctg agtggggact ccttttatgg caagagagaa |
| 4441 | ttgacaccac ttgctcctgg aacttctggg gcaattacaa aggatccatt gtatctaaat |
| 4501 | cctcagtacc tctaaaggat atcccatcgg gtagtgcccg gaatggatat tgggctttga |
| 4561 | gcaatgatga agttcaagag attgatcatg tcccttacaa cttgagatat tattgttact |
| 4621 | ggtgcagaaa tgaatatcct gggagctttt atatgagata tgtaaagaaa gttcggatca |
| 4681 | taagaaatcc tgatgggtct ataaagactc ctagaggatc ctgggttcat gagttggaca |
| 4741 | acttgtgggg agatcagatg aggtatctag ttattcgaag atttggggga gaatctagct |
| 4801 | gccctcttaa gatatatgat gtgagagcag gggttctgtc aaaatctcgg tcaaacttca |
| 4861 | tcttagtgtc ccttccctcc ttgaatttgc agttctctgt atcacttgaa tccactgaga |
| 4921 | cgaaatgctc atttggagat aagacatatg atattgtgca gagcatggga ggctatctcc |
| 4981 | tctccatcga cataggtaat gcgaactggc gaggcccttg ggatcctacc cctcagcatc |
| 5041 | cgggtcgtga aagaagatca attatggagt ttccggatca aacatctttc agatataacc |
| 5101 | aatttataaa ttatcactca tccccaagac acaagagaca tgatcaagaa tttgagttcc |
| 5161 | ctctcagtct aaaatccagt tatgattatg ctcaatttag atatgagcag aatttcatca |
| 5221 | tccgacagat caataagaat tttggattat tacagaagag catttgtgat attcagtttt |
| 5281 | ctaagtggca gaatctcagt ccacccaatc ttgctatgaa aattgcccat tatgtcaccg |
| 5341 | gctctatcca ctctataggt ggtgttcatc atggatctta ttcaattcaa agaacggaaa |
| 5401 | aatccattac taaggtcaat ctggtgtttc ccattgttat tgttcatgga atgtataagt |
| 5461 | gccaaaggga accatccaag gaggtggttt gggcagaacc cgtcacaggg atcttattca |
| 5521 | agtctcctat tccgactcat ttctcactaa gttcctcttg gctacctggg gtaaatggtt |
| 5581 | cttctattgt ccctctgaca ggtcaaattc ttctccctga aatcacaatg gatcacttgg |
| 5641 | aggttgtaca acaggttgaa gcaaagatgg tcaaaagtat gtacacgaat gtagagttgt |
| 5701 | ttggatcaac agaggaattt caaagatacc aaactcaggg aattacctct gatgaacaat |
| 5761 | caaatacagt aaatccttgg attgggcttt tgatacatgg tggagtgtcc atagctactg |
| 5821 | gaatattagt agcacttttg atcccctcaa tcttaaaatt gttcagacat ataattgaga |
| 5881 | aaggggaggc atcgttagag gagaggttgc atctgaggga aacctcaaga aaagaatttg |
| 5941 | tcaaggttag ggggaaacca tggggtgtct aagctaccac agcttccaca agagattgga |
| 6001 | ctccaggtgg ctctctccat caagcgatca tgactcacaa agtcccttca ggccctaaca |
| 6061 | gatcagtaca gccatcattc atttgggctc catctggccc caaccgactc ctcttataaa |
| 6121 | aaaccaatga aacttataca gatcgtcctt gactgccaaa atggatctca ctttagacac |
| 6181 | tatgaggcat attgaaactc tgatcaattc acatctagag cttgaagacc ttaaatcttt |
| 6241 | gattacagac acttgtttga ttcactcaag ggatctatac aatcccttct tatacattat |
| 6301 | ctgttttgtc aaacctacca tcacagccag tgctgaaaac tttatgattg ggaaattaaa |
| 6361 | gaagatcata attccattct gggatgtggt tgatgtcaca agatgcaaaa gaatcatttg |
| 6421 | tactgaattt gctccagatg atgtgatatt aatgaaactg acccctgtga tctcttatca |
| 6481 | ctctgcataa taaccagatc tctaaatata ttttaaaggg atagaaaaat ccttccctag |
| 6541 | ttattagttt ataccagtgt ctttatttat atttaatcta agatttcttt atagtgattc |
| 6601 | caattagaga agggatgaac tttagactta ttggttgtga tatagtaatg aaatagacag |
| 6661 | ttatttattc ttagttaatc tttaaacatt ttccttcttc tatttttcgg ttaagtcacc |
| 6721 | aaactaccta tcaaaccaat acatgagaac agtgtattat tcctgctatt aaaaaagatc |
| 6781 | ataccttttc catctcagct cctcagtcaa atcttagttt cattaaatca ccatggatga |
| 6841 | attacaaagt gataatgtcc gtaaaaaacg tcccccttta tcaactcatt gtgacacccc |
| 6901 | tctcaccctc aacaatgcca gaaaagcctt attagtacct gcacccggtc aattcataca |
| 6961 | tcccaataac cccattcgac gagagtactt ggagatgcag agacaacttc agataacacc |
| 7021 | ccccaatcta tttgatctat caaaagttca gggttttttc ctaaatgtgt ttaatgtacc |
| 7081 | agtctctagc cttcctttat tagaatttag acaagcattg cacttggctt ctcaactata |
| 7141 | ccaagtagaa gttgaagggg ttctcaaaga gctaggggca tcagctacta aaattgatat |
| 7201 | atctcctctg atgaaaaata aggacttaat taatctttat ctgagaaaat gtttctggga |
| 7261 | ggaagcagtt gtcatgagtg gaaatgataa ctctagtcag ggatcctggt ggtcaagagc |
| 7321 | agataaaggg cttattctct ttagacgacc tgggcttgat atcataattg gggagaattt |
| 7381 | aatgtcaatc cagacatctc agaactccat attggtctcc cgagatcacc taaccatatt |
| 7441 | gtcagatctc gctgctgagc ggtttagtat aattctccaa tccttcttag ctgatcaaac |
| 7501 | ccataataca gatatgccca ccccttccga attaagttta tttcttaagg aaggagatga |
| 7561 | aatgctaact ttagcaggaa atcaaggata tgatctaatt tatactttag aatcttcctg |
| 7621 | tacttcccga ttagtaggga actatgaagg aggctcgtgg aaagattcta aattccggca |
| 7681 | tgaaattgtt aaagatttag agaaaaaagc ctcagatcta aatttacatc ctcaacttag |
| 7741 | agttagagaa caactgttag attcagtttt tgaacgaaat ctaaacgcct tcacccaact |
| 7801 | gtatgggcta tatcgcatat ggggtcaccc aactctggat ccattacttg ggacaatagc |
| 7861 | cctcaaagaa ttgggaacaa caccaagatt gtacctatca caccaagctc aggagattaa |
| 7921 | caacaagttt aaggaagagt tcataaaaag atatttaaat agacataagg agtggccgga |
| 7981 | attagatgta tcgaaattac caagacataa catcattcga gtccattatg agaagaaatt |
| 8041 | acaatttcct tctaaatcca gacaatatag gagatctcat ctctccttgg tagaattcaa |
| 8101 | agaggtattc cctgttgatc ctaaatttga tcttattgaa tttattgatg ataaatccat |
| 8161 | ctccttaggt ttcccagatc tccttaacga gatctataga aacaagagta tcgggaattc |
| 8221 | actagcaaga tccttattgc ttaatttcct ctcctctgac atttcagacc cccaagaatt |
| 8281 | tctgaagaat atagatacct cagggtttcc tcctgaagag atttgtgttg gggtacacga |
| 8341 | aaaagagaga gaaggaaagc taaaggcaag gctgtttgga ttactgacct tagtgaaacg |
| 8401 | atcatatgta gttatcacag aaaaactctt ggctgagcat ctatttccgt atttccctga |
| 8461 | aataaccatg acggatgacg agttagtttt ggagaaaaag aggcatgcat tcaatacaga |
| 8521 | acgaaaaaac aaatttatgg tgagtttgga tttttccaag tggaacacca atatgagagc |
| 8581 | cccagacaca cagccatttt accacactat agatacgatg tttggtttgg aaaattgttt |
| 8641 | taccaggaca catgaaatgt tctacaattc ctttttgtac cttatagacg gttcttatct |
| 8701 | cccaacaata gttgatgatg ggttcaaaac agatattgga tgttggcgac atcatcttgg |
| 8761 | gggaatcgaa ggtctcagac aaaaaggatg gactctgtgg acagttatgt tgatcaggct |
| 8821 | agttgcggaa aaatatattt tcaatatgtc tatcatggga cagggggaca atcaaatgct |
| 8881 | acttctaact ttcgattcta ataccccgga agaatatgcc ctctctcaag ttaatgattt |
| 8941 | ccttcagtca ttaaaggata aactgtcact aataggtcct cctctcaagt tggaggaaac |
| 9001 | ttggatttcc aaagactttt atttatatgg aaagtatcct atcaaaggag gtgtttctct |
| 9061 | caccacatcg tggaaaaaat catgcagaat gttccgatgt tgcaacgagg actatcccac |
| 9121 | catagagtcc agtttgtcct ccttagctgc aaacctgtac tctgcagtgg ctgctgataa |
| 9181 | ctttacacag actctgtttt ttgtttactt atttgaatta gtaggtctat tccaatgcaa |
| 9241 | tattagaaga ccctatctcc aaaagaactc attttatcaa tcgttagatc gaaatagaac |
| 9301 | cttcacagtt gcttctgcaa aagaccaaaa gaagaaactt catgtccctc ttgttctatc |
| 9361 | acccccaaat cagctacagc ctaccgaggt tttgttagga ctatgtttga ctccgaggac |
| 9421 | tttgggagga tatccagttg ttctgtaccc atcggtcttg ataaagggag ccccagacca |
| 9481 | attatcattt gatcttgcgt ccttaaaatt attttcaaag tcagcagatg caactgttaa |
| 9541 | taggataata acccgtgtat ccagtccatt cctctccgag tataagaatt attctctact |
| 9601 | ttttatgaac cctgaggcaa ttatcctgga gtctacaccc actcctgcag aggcaaggag |
| 9661 | aactacgatg ttagaatttc tttccaacag tgatcgtgtt aaccagcctt acataaaaga |
| 9721 | attcctaaac atcattcatg agaatgcaaa tcaatctatg gaagattttt taacctcaaa |
| 9781 | tcctgtactt catccacgtg taatctctct tctacttcag gcaactccac aatacagagc |
| 9841 | tcaacaagta attggaaggc tccaaaagac cccaacaatg gctagagtct acttaagaga |
| 9901 | gggagataga gatctttatg cactgttaga gatgtctgag ttgaatcatt ttaagtcagt |
| 9961 | attacgacaa gtctttgctg aggtaggcag gtattcattg cctcacttca actctttggt |
| 10021 | tgaacattcc acctttttaa ggaacatggg ttggggtaaa ataattgaag gtgtagatag |
| 10081 | tgcccctcct catgaggtct tccatctaga ggtcatgaca tctattacag aatgccagga |
| 10141 | ttctccacat gcagacctgg ggttcatatc agttagactg aacaccccta aagatgttgc |
| 10201 | aggaaattct cttgcaattg gaaccacaag accttataga ggatctatca ctaaaaataa |
| 10261 | agtcaactcc ttatcaacaa aaatccaagc cagaacccct tctctgttac aaagagctct |
| 10321 | catggttgca ggactggaat cctgggcatt cactaaggat tcttcacttg ctcagttatc |
| 10381 | aagggggtta gtttggagtg ttacagactt accgtatgaa ctgttgactc cacaggtaga |
| 10441 | tcaggtttcg ggatcatatc aacatcgctt acgaaatgat agactagaca atgggggaat |
| 10501 | cagtcctgtt ttaccgaatc aaggtaccaa attacaattc aacactgtcc ctcttgtatg |
| 10561 | tttgaataaa ggaagtaaaa ataagaatgt catgttccaa gggcctttag tgatgtttgg |
| 10621 | aagtgtaatt ggcgagggac tgcttacaga aggtatcaat tatccagaga caaaactatt |
| 10681 | tcacatccat attaggaatc cctcttccat acaggatctt gatgaaaatc caatcaccia |
| 10741 | tccccctatt caacagccta tcagattgtt acgaaacccc caatctcctt tcttattttt |
| 10801 | cccatctgac aaaatcatgc cttatataaa aagaattcta aaatatccta tctgttcacg |
| 10861 | ggaagatctc aatgtgattt ctacagaatc tcgcttcaat actttattgg cctacgagtg |
| 10921 | cataaacttg cttgatccat ggtcatgggt ctctggatct gactcgagat tggtaactaa |
| 10981 | cggaataacc atcaactggg ctctctcttg taatattgta gagctatgtc ttgtgatcag |
| 11041 | cctcttactt ctagctattt tcttcacccc aactaaaatc attgatccgg aatggcatat |
| 11101 | caatagagtg ataaatgttg tcaaagcttc ccctctctcc tcatgggaga atttaacaaa |
| 11161 | tctctgtttc tgcaatgtct tcccaacccc tctattacat tttcttagag ctatgagccc |
| 11221 | acaaacctct gagggtttaa caaattcaaa tgttgcactt atactaaaga cttccattac |
| 11281 | attgatcctt cagaatatcc tttacgatag gaatttcata aaaggaaaag tacctcacct |
| 11341 | gattgcaccc ccggctgtgt cttttaacct gcacccttac agggtcatag aaatcctagg |
| 11401 | gtggttatat aaggaacatg aagttcataa atcccatctt tcgtacctct ctaaggacat |
| 11461 | gctagatttg aaacttagga gtttggaagg tccctatgtt caccaattag acttttgggg |
| 11521 | gagccctacc cgaatatctg gaatcagctc tgagtctctg gattatcttt gtaaattgga |
| 11581 | agacgtaatt aaatctagat ccatagaggt actcacagtt gctacaatat ttgatccaaa |
| 11641 | tccactccca atgcctctag ttactggccc cattgttaag tcgggtgttg tgaacacaag |
| 11701 | gctgcaagta tctttttatt cagaggggga cgtactgcat ccggaattag gacatagaga |
| 11761 | ttatagaact ttctttttcc gaccagctcc attgcccaca tcaggggctt ataagctatc |
| 11821 | aagtgttttg cccttactag gaagctgtgc cttgacaaaa tgcctctgtt tagctgatgg |
| 11881 | aacaggtgga ttcaccagaa cactcgctct tagagatgat tcgaaaacaa ttgtattcaa |
| 11941 | tacattagta actgatcaag attatgtaac acaaatggac ccaattcaaa atatcccaga |
| 12001 | cattgcagat cttccaattt cttgtcagaa gaaagttgtg ggcctaaggg aggtcaatga |
| 12061 | ttatccaaca gatataacaa gcccggattt cggacatcaa attctggaca gatttggagg |
| 12121 | tgatttcatc ttggttacag gagatgcgga agacccctct ctgcaccata gtgggaatgt |
| 12181 | tttagctttg ttcaaagcct atatggatat tagcctaatt gtgaattccg ttcatggtat |
| 12241 | ctttaagatt catactcaca gaaggtctgt cctacatcag gctctggtta tattgttgac |
| 12301 | ttattatgac tctgtagttg tagtaagaag tcaattctct ctccgatcca acaatgaatt |
| 12361 | ttatcttgtt ggtgcaagaa acaagttagc tcctaaaatc cttccattaa atattattac |
| 12421 | tctggcagat ggtaatccga ccctaaatgt tggtctaagc agagatgctg aactgatgct |
| 12481 | gaataaaagt ctcaacaact taaatagaca acaaggtcaa ctgcaagagc tacaaaaatc |
| 12541 | tacctacata cagattacat ccaacttaat gcctcacctc cattaccaag atctaatctt |
| 12601 | tcacctagtt ggtcagtatc catggttgaa acaggaatac tttgatatcg gggaatccaa |
| 12661 | ggatggggat ctcagaccta tctactcatc ttgtatccat aagtttgtaa cctctataca |
| 12721 | caaggcaaat accaaatatg aggagaaaga tgtagcaaga tttgtggtaa agcaatttac |
| 12781 | tctaagagag ctgtcagata tattatcctc ctatctcttt ctgatactgt ctgttcttcc |
| 12841 | cctagccaga tggaattctt ggatacctca cttcttgaaa gaaggatgct tgttatggat |
| 12901 | acaatgtgag aatgggttgt ggttcttttc tccctatctg ggggttgtcc cacctgcgag |
| 12961 | atcaacctat cattttagac tgtataggac acaagattta ttaaatcatg ttgcgatcca |
| 13021 | gaggatttgc agatctgttg gtttagctca catgttacac ttcagggaac ctgacgataa |
| 13081 | acatcttaaa gaagagtcca tctttactag accatcaagg aaattcacat tctatgatcc |
| 13141 | taaactcaag ggactaaaag acaagattaa atgtcagagt tggcaatggc tcagtcaatc |
| 13201 | tcctggttat caactggatc agtttgctcg aatttcccaa aatcccaaga aataaggagc |
| 13261 | atctatcaaa tcccccgacc aatgattgtc tctttgtcct ccatcaaact atatataaat |
| 13321 | caatcaatcg tcttaaagtt ctcttgattt ttgttgtata gattataaaa aaccaattat |
| 13381 | tttaattact tctctcattt acagttagtg tcccttaaga atatctggct tcatgtcatc |
| 13441 | aagggtggac cctctatttt atcttcattt tctctcagca acccactgca tcaatcattt |
| 13501 | cttcccctgc ttgccccccc tcccccacga tcta. |

### Biological Deposits

The Applicant requests that a sample of the deposited microorganism should be made available only to an expert approved by the Applicant.

Spodoptera frugiperda cell line identified as 281G3 deposited with the American Type Culture Collection (ATCC), P.O. Box 1549, Manassas, Virginia, 20110, U.S.A. on May 5, 2023, under ATCC Accession Number PTA-127562.

Spodoptera frugiperda cell line identified as 282H10 deposited with the American Type Culture Collection (ATCC), P.O. Box 1549, Manassas, Virginia, 20110, U.S.A. on May 5, 2023, under ATCC Accession Number PTA-127563.

Spodoptera frugiperda cell line identified as 284C2 deposited with the American Type Culture Collection (ATCC), P.O. Box 1549, Manassas, Virginia, 20110, U.S.A. on May 5, 2023, under ATCC Accession Number PTA-127565.

Spodoptera frugiperda cell line identified as 281G3-2 deposited with the American Type Culture Collection (ATCC), P.O. Box 1549, Manassas, Virginia, 20110, U.S.A. on May 5, 2023, under ATCC Accession Number PTA-127566.

Spodoptera frugiperda cell line identified as 284C2-2 deposited with the American Type Culture Collection (ATCC), P.O. Box 1549, Manassas, Virginia, 20110, U.S.A. on May 5, 2023, under ATCC Accession Number PTA-127567.

Spodoptera frugiperda cell line identified as 281A5 deposited with the American Type Culture Collection (ATCC), P.O. Box 1549, Manassas, Virginia, 20110, U.S.A. on May 5, 2023, under ATCC Accession Number PTA-127568.

The deposit was made according to the Budapest treaty on the international recognition of the deposit of microorganisms for the purposes of patent procedure.

What is claimed is:

1. A Spodoptera frugiperda cell line deposited as accession number PTA-127562, PTA-127563, PTA-127565, PTA-127566, PTA-127567, or PTA-127568.

2. The cell line of any preceding claim, wherein the cell line is suspended, stored, or maintained in a preservative, cryoprotectant, solvent, buffer, media, antimicrobial agent, antibacterial agent, antifungal agent, or other agent. 3 The cell line of any preceding claim, wherein the cell line is free of Sf9 rhabdovirus.

4. The cell line of any preceding claim, wherein the cell line is free of nucleic acid sequences having at least 70%, 75%, 80%, 85%, 90%, 95%, 98%, or 99% homology to the sequence set forth in NCBI Accession No. KF947078.

5. The cell line of any preceding claim, wherein the cell line is lyophilized.

6. The cell line of any preceding claim, wherein cells of the cell line are diploid.

7. The cell line of any preceding claim, wherein cells of the cell line are engineered to produce a recombinant biological product and/or to enhance production of a non-recombinant biological product.

8. A composition comprising a Spodoptera frugiperda cell line selected from the group consisting of cell lines deposited as accession numbers PTA-127562, PTA-127563, PTA-127565, PTA-127566, PTA-127567, and PTA-127568.

9. The composition of any preceding claim, further comprising a preservative, cryoprotectant, solvent, buffer, media, antimicrobial agent, antibacterial agent, antifungal agent, or other agent.

10. The composition of any preceding claim, wherein the cell line is free of Sf9 rhabdovirus.

11. The composition of any preceding claim, wherein the cell line is free of nucleic acid sequences having at least 70%, 75%, 80%, 85%, 90%, 95%, 98%, or 99% homology to the sequence set forth in NCBI Accession No. KF947078.

12. The composition of any preceding claim, wherein the cell line is lyophilized.

13. The composition of any preceding claim, wherein cells of the cell line are diploid.

14. The composition of any preceding claim, wherein cells of the cell line are engineered to produce a recombinant biological product and/or to enhance production of a non-recombinant biological product.

15. A kit comprising one or more Spodoptera frugiperda cell lines deposited as accession number PTA-127562, PTA-127563, PTA-127565, PTA-127566, PTA-127567, or PTA-127568.

16. The kit of any preceding claim, further comprising a preservative, cryoprotectant, solvent, buffer, media, antimicrobial agent, antibacterial agent, antifungal agent, or other agent.

17. The kit of any preceding claim, wherein cells of the cell line are suspended, stored, or maintained in a preservative, cryoprotectant, solvent, buffer, media, antimicrobial agent, antibacterial agent, antifungal agent, or other agent.

18. The kit of any preceding claim, wherein the cell line is free of Sf9 rhabdovirus.

19. The kit of any preceding claim, wherein the cell line is free of nucleic acid sequences having at least 70%, 75%, 80%, 85%, 90%, 95%, 98%, or 99% homology to the sequence set forth in NCBI Accession No. KF947078.

20. The kit of any preceding claim, wherein the cell line is lyophilized. 21 The kit of any preceding claim, wherein cells of the cell line are diploid.

22. The kit of any preceding claim, wherein cells of the cell line are engineered to produce a recombinant biological product and/or to enhance production of a non-recombinant biological product.

23. A method of making a diploid Sf9 cell line free of Sf9 rhabdovirus, the method comprising the steps of:
subjecting a suspension of Sf9 cells to segregation using an automated cell sorter to obtain one or more single-cell isolates;
maintaining the one or more single-cell isolates under conditions that promote cell growth and division to obtain one or more clonal cell cultures;
expanding the one or more clonal cell cultures under conditions that promote cell growth and division to obtain one or more clonal cell lines;
subjecting the one or more clonal cell lines to molecular testing to detect the presence or absence of Sf9 rhabdovirus; and
subjecting the one or more clonal cell lines to further molecular testing to detect the ploidy of the cell line.

24. The method of any preceding claim, wherein the automated cell sorter is a cell printer.

25. The method of any preceding claim, wherein conditions that promote cell growth and division comprise the use of standard cell culture media.

26. The method of any preceding claim, wherein conditions that promote cell growth and division comprise the use of conditioned cell culture media.

27. The method of any preceding claim, wherein the molecular testing comprises the detection of Sf rhabdovirus nucleic acid sequences.

28. The method of any preceding claim, wherein the molecular testing comprises PCR amplification of Sf rhabdovirus nucleic acid sequences.

29. The method of any preceding claim, wherein the further molecular testing to detect the ploidy of the cell line comprises karyotyping.

30. The method of any preceding claim, wherein the further molecular testing to detect the ploidy of the cell line comprises quantitative PCR.

31. A method of making a biological product free of Sf9 rhabdovirus, the method comprising the steps of culturing one or more Spodoptera frugiperda cell lines deposited as accession number PTA-127562, PTA-127563, PTA-127565, PTA-127566, PTA-127567, or PTA-127568 under conditions that promote production of the biological product, maintaining the culture for a time sufficient for the accumulation of biological product, and isolating the biological product.

32. The method of any preceding claim, wherein the biological product is a recombinant biological product or a non-recombinant biological product.

33. The method of any preceding claim, wherein the biological product is an RNA, a DNA, a polysaccharide, a lipid, a peptide, a protein, an enzyme, an antibody, or a vaccine.


SEQUENCE LISTING (21 sequences)

SEQ ID NO: 1 - DNA, 13534 bp, unidentified
  source 1..13534: mol_type = unassigned DNA
tgaaaattatacgataaatgatcctaactcttatagatgacaacgactacttcacggatatcggcttcttagatcttatcatgacacagggaaccatgaagccagtatgggaagaattggggacaggagaaacagagttccaagggaccgtggacattccagggagatctctcaagccagaaaaaacagattggagtgttgatacatgtcgggagatcagtttaaatctgaagttacctggtgaaatatggcaactggcccatcaagaaaccatcttcaacagatttcttacattttacgctactgggtatgttccaaatacacacacagccacagaaattgtactctccatggcatcactaatcttcaaggacaaggccaaagcacctattgatttgatttgggatgactcatttcaagctagtccctctgaggagtgtgggttctccgttgttggagaaactccattggttatcggacaacacccggatgatgatgactacacattgagagaagatgaagaatcagccgctatgaatgaggaagaaaaaatacaagcagctctaaaaactttgggaattcaagatactccagtagacctgaaggatgcatctggaattgtctttgagacaaaggaggacagagaacaaaggatcaagaatgagaaagctctacatgtagaggatgatatcaacgctctaactcagattacaaaacaattcttgtttgagtattccacaggctccctacagaaatttgttgcaaaggctactactattttcatagataataatgctactaacggcttcacccgtttgcatctccatgccatcagagtcatgaacttcattgctctaacaatgcttagaaaggtaaccaagtcaaatgcccagatgatcaatgcctttctgaaggagcaatacaagagaaatattgcctccctaatccccggcgccctctcctctgattttgctcctcccagtaagagctgcattgataaactgacagctatttctaagaatgacccggcagtcagttcattctttgcaaaggttgtgatgctcaacatggaggaggaacggagaaacccttctctggttgcttgtcttggggcttcccttctcacccacaccacttggaatggaatggggattttacatgttatttttgaagtttgtctattccatcagattagctggaagaggttggtcacagagtccctgacctcactaacaaagatgtcatggggtgaagtcagtcaattcctcatcaagtatcaagcaaagggaaatcctgacccaacggttgcctgggccagaatcattgatgattcttactttatgagattaaccatagtaaatcatcccacacttgctgcattattagtggaatccctcataagatctcagaaagatgatggaatcctgaatgccaactgggccatccaacacagggacaccatcaattattatagggacgctgccaagcttctcactgataagctcacaggacagactgctacagtccaagcccttaccaatgaagccgctgatctagttagaacaatgaatgcaggaccctctagataccacccaaggcctagtacccttatccccatggtagatctaaacccggaagacttataagacttacctattatcccaagactaatttccataataatccccaaaaagacaattactgttattttctattaaaaaaccaatgaaaattatgcagagaatattgagacatatagtatccttcttctcaaagtcctggtgccaatcctccgatcccgctctagtgtgcgactgtgagtatcctcctctcaagagaaactatcaactgatttactctataatggcttcccactctcttgacaccattgatctatctgaaattggattgacaagggaggttctgactggggttggcgattacatgactggacaaagaccggttccagccttcaatcctccagaggtcggtcactccccctctgatgaagtggcaaaacgattgggagaactgaagaattactggactcagttagaggatcctcttgatgagagaattctcaataccttgaaagcgatcagcatcctgagcggagacaccagaggagatctgagtggaaaatataaacatctagtccgcattagcggagatgacatgccccaattattggacgaacttatagacatctgtcttctggggcctaagactctaattgctaccttacgaatggcgataaccgcctataccgctgcattagccagaaatgccaagtccaccatctcagatattactaccgcatcagcagatttgatggtcatcactcagatgatacagtcccagcaggaatctttccaatcatcattagagcatctctctcatgcttggaataacgtcgccagtggtatgactgcttatacagcggaactggataagagaaccctcaagttgacacagcttacaccgccagtcaagcctgaccatcaccgggccccctctacagcctccagtcatacccctgatgcttcagtggggcttcatataaacataacccccggttgtgcatataagtcccaatttggggtattgacttgctgtcccaatgggaacattggttttcttgcaaataactccgatggcactgtgatagccaaacttgtggaagtaattaggaagccacgccctctcacgacggccctcaataagaacctgtctgagctgatcaattatgtcaaggcaaatccgaagatccttgctacttactctacctcttctcctcaggataagcttaatatcctgaatgagatccacttctgcatcccagatcttaccaatcaatgggtcaaggcctgataacattcccttccctcacaaactctaaagatatctccatccttgatgacatttttattaattatatttaagtacgatttaagatctacataccaaaaccaaccattttgatgattaacatgttattataaaaaaccaatgaaacttatacgttaattgagataagtttgatttcattgttccttgtgtcatcaccgaactttttgagctcgaacaatgagtgctcttgaacggattgcacggagcctatcattgaagaagttgaaccctaggagaactccaaagactcagcctattcctgaaaaggctactgtctatcatcctttcatgctctcttatgatctcaatttagctattgagggtaaaatccacatctccgctatcaccattatagtgaatgccctttccttagcttgggcaatagaactctttcactctgactcatcgtggtcagggtgtcttgagtacttttggaaatccatcaaggataacatattggcatccataaaccctcgagttgatccaaatggaacttgtcatatgatgacatcaatcataactttcctcggattctcagatggatcctgcatcaactcagaagcagagccaagacagctcacaggatctagatcctgggagatcatgtctcctaatcagaatctcattgtgataaccctaggattcaaaataaccttgaaaaccttcgcacagcaccagagatacagcttgcgtgaccatggattccacaaattggagatgctcaacgagaaagagaagaaaatgttgaactatatgggggtcaaacaattaaaaccccagtatacacatgaaaagacattcgagaaactcattctcaagaacaaaggtccaaaggggtctcgtgtcagggcaattcttcactctcaaagtcgtgacatgtggtctccaaccgctccttctcctccacccacatatgaagatggatcctcagatgaatgggatcagcaacaactgcacagcctcaaccacctgcatacaccttctgtccccctgagggcccccaggacatccccaccccaacaactctccccaaaaccgacatccacaacccaacccctcccacaactcacacaaccaaacaagccccaagaactctccaagtagacacttgacagcccccaccaattacttttagatcataaaaaaccaacaggcagaatataagacctatcaattagagatattaaaaaatactaattaaacaattatacatcaagcattggctcattatggttttcttaagtttatcaacgatcatatttatcctaagcctccgggctgtaacctgctccaatcctctctcctatcctaatggcattttgactaacaactctactcacaatcatcccctatcggacttttatattttttatgagaacagttcccttacctatactcaattccctgtggccccagactgctctagtattctagatactagagatgagcagtatcccaccactgttactttgtggaaggttgatcaagaatctcaagctgagtggggactccttttatggcaagagagaattgacaccacttgctcctggaacttctggggcaattacaaaggatccattgtatctaaatcctcagtacctctaaaggatatcccatcgggtagtgcccggaatggatattgggctttgagcaatgatgaagttcaagagattgatcatgtcccttacaacttgagatattattgttactggtgcagaaatgaatatcctgggagcttttatatgagatatgtaaagaaagttcggatcataagaaatcctgatgggtctataaagactcctagaggatcctgggttcatgagttggacaacttgtggggagatcagatgaggtatctagttattcgaagatttgggggagaatctagctgccctcttaagatatatgatgtgagagcaggggttctgtcaaaatctcggtcaaacttcatcttagtgtcccttccctccttgaatttgcagttctctgtatcacttgaatccactgagacgaaatgctcatttggagataagacatatgatattgtgcagagcatgggaggctatctcctctccatcgacataggtaatgcgaactggcgaggcccttgggatcctacccctcagcatccgggtcgtgaaagaagatcaattatggagtttccggatcaaacatctttcagatataaccaatttataaattatcactcatccccaagacacaagagacatgatcaagaatttgagttccctctcagtctaaaatccagttatgattatgctcaatttagatatgagcagaatttcatcatccgacagatcaataagaattttggattattacagaagagcatttgtgatattcagttttctaagtggcagaatctcagtccacccaatcttgctatgaaaattgcccattatgtcaccggctctatccactctataggtggtgttcatcatggatcttattcaattcaaagaacggaaaaatccattactaaggtcaatctggtgtttcccattgttattgttcatggaatgtataagtgccaaagggaaccatccaaggaggtggtttgggcagaacccgtcacagggatcttattcaagtctcctattccgactcatttctcactaagttcctcttggctacctggggtaaatggttcttctattgtccctctgacaggtcaaattcttctccctgaaatcacaatggatcacttggaggttgtacaacaggttgaagcaaagatggtcaaaagtatgtacacgaatgtagagttgtttggatcaacagaggaatttcaaagataccaaactcagggaattacctctgatgaacaatcaaatacagtaaatccttggattgggcttttgatacatggtggagtgtccatagctactggaatattagtagcacttttgatcccctcaatcttaaaattgttcagacatataattgagaaaggggaggcatcgttagaggagaggttgcatctgagggaaacctcaagaaaagaatttgtcaaggttagggggaaaccatggggtgtctaagctaccacagcttccacaagagattggactccaggtggctctctccatcaagcgatcatgactcacaaagtcccttcaggccctaacagatcagtacagccatcattcatttgggctccatctggccccaaccgactcctcttataaaaaaccaatgaaacttatacagatcgtccttgactgccaaaatggatctcactttagacactatgaggcatattgaaactctgatcaattcacatctagagcttgaagaccttaaatctttgattacagacacttgtttgattcactcaagggatctatacaatcccttcttatacattatctgttttgtcaaacctaccatcacagccagtgctgaaaactttatgattgggaaattaaagaagatcataattccattctgggatgtggttgatgtcacaagatgcaaaagaatcatttgtactgaatttgctccagatgatgtgatattaatgaaactgacccctgtgatctcttatcactctgcataataaccagatctctaaatatattttaaagggatagaaaaatccttccctagttattagtttataccagtgtctttatttatatttaatctaagatttctttatagtgattccaattagagaagggatgaactttagacttattggttgtgatatagtaatgaaatagacagttatttattcttagttaatctttaaacattttccttcttctatttttcggttaagtcaccaaactacctatcaaaccaatacatgagaacagtgtattattcctgctattaaaaaagatcataccttttccatctcagctcctcagtcaaatcttagtttcattaaatcaccatggatgaattacaaagtgataatgtccgtaaaaaacgtccccctttatcaactcattgtgacacccctctcaccctcaacaatgccagaaaagccttattagtacctgcacccggtcaattcatacatcccaataaccccattcgacgagagtacttggagatgcagagacaacttcagataacaccccccaatctatttgatctatcaaaagttcagggttttttcctaaatgtgtttaatgtaccagtctctagccttcctttattagaatttagacaagcattgcacttggcttctcaactataccaagtagaagttgaaggggttctcaaagagctaggggcatcagctactaaaattgatatatctcctctgatgaaaaataaggacttaattaatctttatctgagaaaatgtttctgggaggaagcagttgtcatgagtggaaatgataactctagtcagggatcctggtggtcaagagcagataaagggcttattctctttagacgacctgggcttgatatcataattggggagaatttaatgtcaatccagacatctcagaactccatattggtctcccgagatcacctaaccatattgtcagatctcgctgctgagcggtttagtataattctccaatccttcttagctgatcaaacccataatacagatatgcccaccccttccgaattaagtttatttcttaaggaaggagatgaaatgctaactttagcaggaaatcaaggatatgatctaatttatactttagaatcttcctgtacttcccgattagtagggaactatgaaggaggctcgtggaaagattctaaattccggcatgaaattgttaaagatttagagaaaaaagcctcagatctaaatttacatcctcaacttagagttagagaacaactgttagattcagtttttgaacgaaatctaaacgccttcacccaactgtatgggctatatcgcatatggggtcacccaactctggatccattacttgggacaatagccctcaaagaattgggaacaacaccaagattgtacctatcacaccaagctcaggagattaacaacaagtttaaggaagagttcataaaaagatatttaaatagacataaggagtggccggaattagatgtatcgaaattaccaagacataacatcattcgagtccattatgagaagaaattacaatttccttctaaatccagacaatataggagatctcatctctccttggtagaattcaaagaggtattccctgttgatcctaaatttgatcttattgaatttattgatgataaatccatctccttaggtttcccagatctccttaacgagatctatagaaacaagagtatcgggaattcactagcaagatccttattgcttaatttcctctcctctgacatttcagacccccaagaatttctgaagaatatagatacctcagggtttcctcctgaagagatttgtgttggggtacacgaaaaagagagagaaggaaagctaaaggcaaggctgtttggattactgaccttagtgaaacgatcatatgtagttatcacagaaaaactcttggctgagcatctatttccgtatttccctgaaataaccatgacggatgacgagttagttttggagaaaaagaggcatgcattcaatacagaacgaaaaaacaaatttatggtgagtttggatttttccaagtggaacaccaatatgagagccccagacacacagccattttaccacactatagatacgatgtttggtttggaaaattgttttaccaggacacatgaaatgttctacaattcctttttgtaccttatagacggttcttatctcccaacaatagttgatgatgggttcaaaacagatattggatgttggcgacatcatcttgggggaatcgaaggtctcagacaaaaaggatggactctgtggacagttatgttgatcaggctagttgcggaaaaatatattttcaatatgtctatcatgggacagggggacaatcaaatgctacttctaactttcgattctaataccccggaagaatatgccctctctcaagttaatgatttccttcagtcattaaaggataaactgtcactaataggtcctcctctcaagttggaggaaacttggatttccaaagacttttatttatatggaaagtatcctatcaaaggaggtgtttctctcaccacatcgtggaaaaaatcatgcagaatgttccgatgttgcaacgaggactatcccaccatagagtccagtttgtcctccttagctgcaaacctgtactctgcagtggctgctgataactttacacagactctgttttttgtttacttatttgaattagtaggtctattccaatgcaatattagaagaccctatctccaaaagaactcattttatcaatcgttagatcgaaatagaaccttcacagttgcttctgcaaaagaccaaaagaagaaacttcatgtccctcttgttctatcacccccaaatcagctacagcctaccgaggttttgttaggactatgtttgactccgaggactttgggaggatatccagttgttctgtacccatcggtcttgataaagggagccccagaccaattatcatttgatcttgcgtccttaaaattattttcaaagtcagcagatgcaactgttaataggataataacccgtgtatccagtccattcctctccgagtataagaattattctctactttttatgaaccctgaggcaattatcctggagtctacacccactcctgcagaggcaaggagaactacgatgttagaatttctttccaacagtgatcgtgttaaccagccttacataaaagaattcctaaacatcattcatgagaatgcaaatcaatctatggaagattttttaacctcaaatcctgtacttcatccacgtgtaatctctcttctacttcaggcaactccacaatacagagctcaacaagtaattggaaggctccaaaagaccccaacaatggctagagtctacttaagagagggagatagagatctttatgcactgttagagatgtctgagttgaatcattttaagtcagtattacgacaagtctttgctgaggtaggcaggtattcattgcctcacttcaactctttggttgaacattccacctttttaaggaacatgggttggggtaaaataattgaaggtgtagatagtgcccctcctcatgaggtcttccatctagaggtcatgacatctattacagaatgccaggattctccacatgcagacctggggttcatatcagttagactgaacacccctaaagatgttgcaggaaattctcttgcaattggaaccacaagaccttatagaggatctatcactaaaaataaagtcaactccttatcaacaaaaatccaagccagaaccccttctctgttacaaagagctctcatggttgcaggactggaatcctgggcattcactaaggattcttcacttgctcagttatcaagggggttagtttggagtgttacagacttaccgtatgaactgttgactccacaggtagatcaggtttcgggatcatatcaacatcgcttacgaaatgatagactagacaatgggggaatcagtcctgttttaccgaatcaaggtaccaaattacaattcaacactgtccctcttgtatgtttgaataaaggaagtaaaaataagaatgtcatgttccaagggcctttagtgatgtttggaagtgtaattggcgagggactgcttacagaaggtatcaattatccagagacaaaactatttcacatccatattaggaatccctcttccatacaggatcttgatgaaaatccaatcacctatccccctattcaacagcctatcagattgttacgaaacccccaatctcctttcttatttttcccatctgacaaaatcatgccttatataaaaagaattctaaaatatcctatctgttcacgggaagatctcaatgtgatttctacagaatctcgcttcaatactttattggcctacgagtgcataaacttgcttgatccatggtcatgggtctctggatctgactcgagattggtaactaacggaataaccatcaactgggctctctcttgtaatattgtagagctatgtcttgtgatcagcctcttacttctagctattttcttcaccccaactaaaatcattgatccggaatggcatatcaatagagtgataaatgttgtcaaagcttcccctctctcctcatgggagaatttaacaaatctctgtttctgcaatgtcttcccaacccctctattacattttcttagagctatgagcccacaaacctctgagggtttaacaaattcaaatgttgcacttatactaaagacttccattacattgatccttcagaatatcctttacgataggaatttcataaaaggaaaagtacctcacctgattgcacccccggctgtgtcttttaacctgcacccttacagggtcatagaaatcctagggtggttatataaggaacatgaagttcataaatcccatctttcgtacctctctaaggacatgctagatttgaaacttaggagtttggaaggtccctatgttcaccaattagacttttgggggagccctacccgaatatctggaatcagctctgagtctctggattatctttgtaaattggaagacgtaattaaatctagatccatagaggtactcacagttgctacaatatttgatccaaatccactcccaatgcctctagttactggccccattgttaagtcgggtgttgtgaacacaaggctgcaagtatctttttattcagagggggacgtactgcatccggaattaggacatagagattatagaactttctttttccgaccagctccattgcccacatcaggggcttataagctatcaagtgttttgcccttactaggaagctgtgccttgacaaaatgcctctgtttagctgatggaacaggtggattcaccagaacactcgctcttagagatgattcgaaaacaattgtattcaatacattagtaactgatcaagattatgtaacacaaatggacccaattcaaaatatcccagacattgcagatcttccaatttcttgtcagaagaaagttgtgggcctaagggaggtcaatgattatccaacagatataacaagcccggatttcggacatcaaattctggacagatttggaggtgatttcatcttggttacaggagatgcggaagacccctctctgcaccatagtgggaatgttttagctttgttcaaagcctatatggatattagcctaattgtgaattccgttcatggtatctttaagattcatactcacagaaggtctgtcctacatcaggctctggttatattgttgacttattatgactctgtagttgtagtaagaagtcaattctctctccgatccaacaatgaattttatcttgttggtgcaagaaacaagttagctcctaaaatccttccattaaatattattactctggcagatggtaatccgaccctaaatgttggtctaagcagagatgctgaactgatgctgaataaaagtctcaacaacttaaatagacaacaaggtcaactgcaagagctacaaaaatctacctacatacagattacatccaacttaatgcctcacctccattaccaagatctaatctttcacctagttggtcagtatccatggttgaaacaggaatactttgatatcggggaatccaaggatggggatctcagacctatctactcatcttgtatccataagtttgtaacctctatacacaaggcaaataccaaatatgaggagaaagatgtagcaagatttgtggtaaagcaatttactctaagagagctgtcagatatattatcctcctatctctttctgatactgtctgttcttcccctagccagatggaattcttggatacctcacttcttgaaagaaggatgcttgttatggatacaatgtgagaatgggttgtggttcttttctccctatctgggggttgtcccacctgcgagatcaacctatcattttagactgtataggacacaagatttattaaatcatgttgcgatccagaggatttgcagatctgttggtttagctcacatgttacacttcagggaacctgacgataaacatcttaaagaagagtccatctttactagaccatcaaggaaattcacattctatgatcctaaactcaagggactaaaagacaagattaaatgtcagagttggcaatggctcagtcaatctcctggttatcaactggatcagtttgctcgaatttcccaaaatcccaagaaataaggagcatctatcaaatcccccgaccaatgattgtctctttgtcctccatcaaactatatataaatcaatcaatcgtcttaaagttctcttgatttttgttgtatagattataaaaaaccaattattttaattacttctctcatttacagttagtgtcccttaagaatatctggcttcatgtcatcaagggtggaccctctattttatcttcattttctctcagcaacccactgcatcaatcatttcttcccctgcttgccccccctcccccacgatcta

SEQ ID NO: 2 - DNA, 25 bp, synthetic construct
  source 1..25: mol_type = other DNA
ggcaaggctgtttggattactgacc

SEQ ID NO: 3 - DNA, 24 bp, synthetic construct
  source 1..24: mol_type = other DNA
acaggtttgcagctaaggaggaca

SEQ ID NO: 4 - DNA, 25 bp, synthetic construct
  source 1..25: mol_type = other DNA
atatgagagccccagacacacagcc

SEQ ID NO: 5 - DNA, 25 bp, synthetic construct
  source 1..25: mol_type = other DNA
acgatgtggtgagagaaacactcct

SEQ ID NO: 6 - DNA, 24 bp, synthetic construct
  source 1..24: mol_type = other DNA
tggcgagggactgcttacagaagg

SEQ ID NO: 7 - DNA, 20 bp, synthetic construct
  source 1..20: mol_type = other DNA
cacagccgggggtgcaatca

SEQ ID NO: 8 - DNA, 29 bp, synthetic construct
  source 1..29: mol_type = other DNA
cctattcaacagcctatcagattgttacg

SEQ ID NO: 9 - DNA, 23 bp, synthetic construct
  source 1..23: mol_type = other DNA
ttgtgggctcatagctctaagaa

SEQ ID NO: 10 - DNA, 23 bp, synthetic construct
  source 1..23: mol_type = other DNA
acaggagatgcggaagacccctc

SEQ ID NO: 11 - DNA, 22 bp, synthetic construct
  source 1..22: mol_type = other DNA
atctcgcaggtgggacaacccc

SEQ ID NO: 12 - DNA, 25 bp, synthetic construct
  source 1..25: mol_type = other DNA
attgtgaattccgttcatggtatct

SEQ ID NO: 13 - DNA, 25 bp, synthetic construct
  source 1..25: mol_type = other DNA
agatgagtagataggtctgagatcc

SEQ ID NO: 14 - DNA, 25 bp, synthetic construct
  source 1..25: mol_type = other DNA
gaacttcattgctctaacaatgctt

SEQ ID NO: 15 - DNA, 20 bp, synthetic construct
  source 1..20: mol_type = other DNA
aagtggtgtgggtgagaagg

SEQ ID NO: 16 - DNA, 20 bp, synthetic construct
  source 1..20: mol_type = other DNA
tgcctttctgaaggagcaat

SEQ ID NO: 17 - DNA, 20 bp, synthetic construct
  source 1..20: mol_type = other DNA
gaagggtttctccgttcctc

SEQ ID NO: 18 - DNA, 25 bp, unidentified
  source 1..25: mol_type = unassigned DNA
atatgagagccccagacacacagcc

SEQ ID NO: 19 - DNA, 26 bp, unidentified
  source 1..26: mol_type = unassigned DNA
acgatgtggtgagagaaacacctcct

SEQ ID NO: 20 - DNA, 25 bp, unidentified
  source 1..25: mol_type = unassigned DNA
acatgagagcccccgatacccagcc

SEQ ID NO: 21 - DNA, 26 bp, unidentified
  source 1..26: mol_type = unassigned DNA
aggatgtggtaagtgaaactcctcct